(19) United States
(12) Patent Application Publication
(10) Pub. No.: US 2008/0171911 A1
(43) Pub. Date: Jul. 17, 2008

(54) METAL ENDOSCOPE HOUSING

(75) Inventor: Harald Hanke, Hamburg (DE)

Correspondence Address: RANKIN, HILL & CLARK LLP, 38210 Glenn Avenue, WILLOUGHBY, OH, 44094-7808, US

(73) Assignee: OLYMPUS WINTER & IBE GMBH

(21) Appl. No.: 11/971,474

(22) Filed: Jan. 9, 2008

(30) Foreign Application Priority Data
Jan. 13, 2007 (DE) 10 2007 002 043.2

(51) Int. Cl. A61B 1/00

(52) U.S. Cl. 600/138

(57) ABSTRACT

A metal endoscope housing designed with longitudinal segments of different cross-sections and/or with a longitudinal axis which may be elbowed or arcuate, is characterized in that the housing consists of separate partial shells which are separated at separation lines running substantially parallel to the longitudinal axis and which are joined in sealed manner to the separation lines.

### BACKGROUND OF THE INVENTION

The present invention relates to an endoscope housing.

Rigid endoscopes comprise metal housings enclosing the inside space in sealing manner. As a rule they comprise an elongated stem adjoined proximally by a main body. In special designs they also might be fitted with an arcuate stem or with multiple bent tube elements, for instance where the ocular is configured at an angle. Such housings comprise over their length longitudinal segments of different cross-sections that may be tubular or other. The tubular segments may be cross-sectionally circular, oval or other.

These endoscope housings are metallic, and because of their complex shapes, present manufacturing difficulties. In general they are built in segments of tubes and other tubular or shell elements that are joined to each other at separation lines transverse to the longitudinal axis by brazing, soldering or the like. This procedure raises problems. Particularly regarding installing the components enclosed by the housing, such as elongated image guides, optic fibers and the like which illustratively must be inserted through the elongated tubular stem.

### SUMMARY OF THE INVENTION

The objective of the present invention is to create an endoscope housing which is simply manufactured and in which the internal components enclosed by the housing are easily assembled.

In the present invention, the housing is divided—at separation lines running parallel to the longitudinal axis—into longitudinally split tubes or shells, hereafter referred to as semi-tubes or partial shells, which subsequently will be joined to each other during assembly. Accordingly, the housing is divided longitudinally into partial shells that may be separately manufactured in a very simple manner. The complex shaping procedure of rigid, whole tubes is thereby eliminated. The assembly of the internal components enclosed by the housing is very simple because they may be laterally laid into one partial shell and then adjusted and covered by the other partial shell. Accordingly, the present invention is applicable both to the external endoscope housing and to any internal housing(s) illustratively enclosing the optic lenses of an image guide system. In the design of the invention, the partial shells may be endowed with complex shapes, a procedure which is far simpler to apply to a partial shell than to a closed housing. Therefore, the present invention offers a simplified way to make housings consisting of a few elongated partial shells of complex shapes, which illustratively present different cross-sections segment-wise, are bent, angled or the like.

The separation lines of the partial shells are connected to each other, for instance, by bonding or soldering. Preferably, they are joined to each other by laser welding, as laser welding provides high mechanical strength and a sealing connection while only slightly heating the sensitive internal components during assembly.

The partial shells may be manufactured in different ways. Preferably, the partial shells are made by compression molding, thereby allowing simple and efficient manufacture with arbitrary shapes.

The separation lines between the partial shells may be shaped in an arbitrary manner, for instance being segment-wise oblique, arcuate or the like for the purpose of e.g., allocating a certain surface detail to a certain partial shell. However, separation lines that run parallel to the housing's longitudinal axis substantially simplify manufacture.

The sectional surfaces may be configured to arbitrarily divide lengthwise the housing into partial shells, the housing for instance when viewed cross-sectionally being resolved into three or more partial shells. According to claim 5 however, preferably the separation lines run along the center of the cross-section, whereby the housing is divided in two partial shells at the largest diameter seen cross-sectionally. In this manner even substantially large internal components which completely fill for instance the housing cross-section can be reliably inserted in problem-free manner.

### BRIEF DESCRIPTION OF THE DRAWINGS

The present invention is shown illustratively and schematically in the appended drawings.

FIG. 1 is a side view of a commercially available endoscope optics,

FIG. 2 is a cross-section along line 2-2 in FIG. 1 of the division into two partial shells,

FIG. 3 is an elevation of FIG. 1 of the housing in an embodiment mode divided into three partial shells,

FIG. 4 is a side view of a commercially available angled endoscope optics,

FIG. 5 is an elevation of the housing of FIG. 4 in an embodiment mode divided into two partial shells,

FIG. 6 is an elevation of the housing of FIG. 4 in a four-fold split embodiment mode,

FIG. 7 is a side view of a tubular housing segment comprising a bulge,

FIG. 8 is a cross-section along line 8-8 of FIG. 7,

FIG. 9 is an axial cross-section of a housing segment having a special cross-sectional contour,

FIG. 10 is a perspective of a curving tube segment divided into two partial shells, and

FIG. 11 is a side view corresponding to FIG. 3 of a longitudinally divided stem tube fitted with a peripheral groove.

### DETAILED DESCRIPTION OF THE INVENTION

FIG. 1 shows a side view of a housing 1 of a straight endoscope optics consisting of a tubular stem 2, a main body 3, a light guide stub 4 mounted on said body 3 and a detachable ocular 5 also mounted on said body 3. Longitudinal segments of the housing 1 are achieved by cutting the housing 1 transverse to a longitudinal axis. As shown in FIG. 1, a longitudinal segment could include the cut segment of the tubular stem 2, the main body 3, and the detachable ocular 5.

The housing parts 2, 3 and 4 are made of metal and are joined to one another by brazing/welding. In the state of the art, assembly in the shown embodiment mode at the separation lines 6 and 7 is implemented by tube lengths 2, 3 and 4 that illustratively may be cut off tube stocks. However assembling such parts is expensive. In particular inserting the inner components which illustratively must be guided through the long and narrow stem 2 is cumbersome.

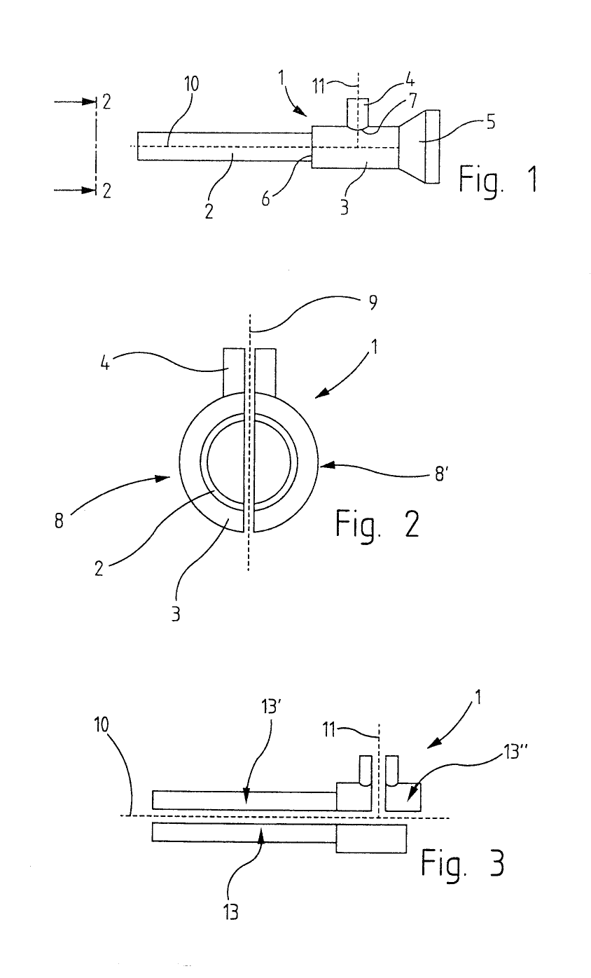

The present invention uses a different assembly method of the housing 1, which in this case is divided into longitudinal, partial shells. In a first embodiment mode of the invention shown by FIG. 2, the entire housing 1 (absent the ocular 3) is constituted by two partial shells 8, 8′ separated along separation lines at a cross-sectional surface 9 which in FIG. 1 is in the plane of the drawing. The two partial shells each contain one cross-sectional half of the stem 2, of the main body 3 and of the light guide hookup stub 4.

In assembly, one of the partial shells 8, 8′ is configured with its aperture upward. Hence all internal components, for instance the image guide and the fiber optics running lengthwise through the housing 1, can be conveniently assembled in place therein. Thereupon, the other partial shell is deposited and the separation lines are joined by, for instance, laser welding.

FIG. 3 shows the housing 1 of FIG. 1 divided into three partial shells. The cross-sectional surfaces 10 and 11 constituting the separation lines run perpendicularly to the plane of the FIG. 1. In this embodiment mode, the three partial shells 13, 13′, 13″ allow very convenient assembly of the internal components, said partial shells being easily connected after abutting one another at the separation lines. The partial shells of FIG. 3, like those of FIG. 2, can be easily and economically produced by compression molding sheetmetal.

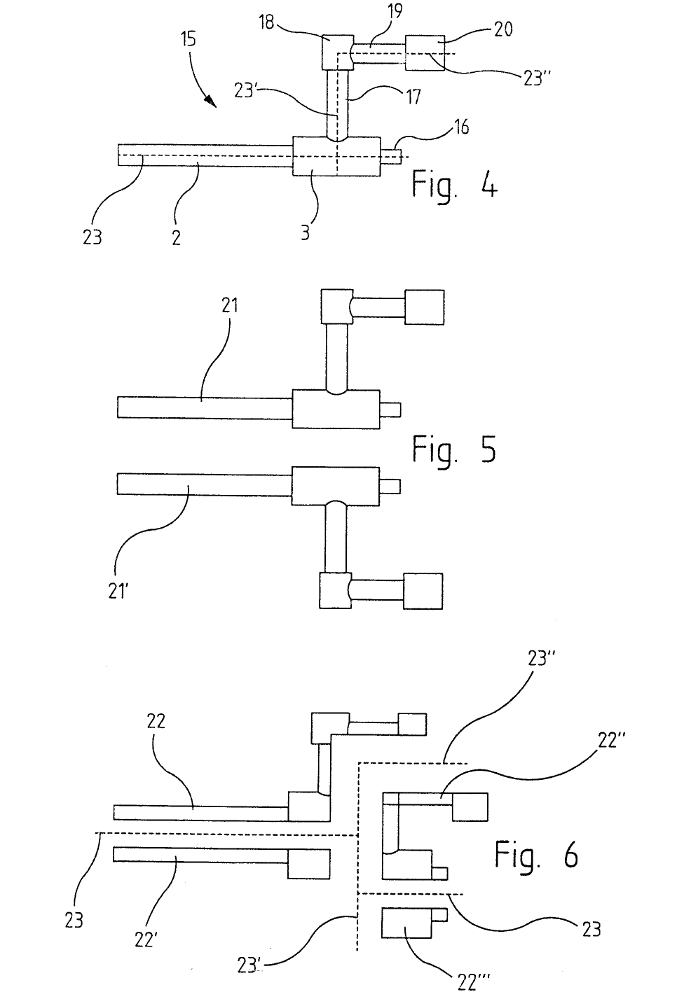

FIG. 4 shows a side view of a housing 15 of an endoscope wherein, as shown, the longitudinal axis follows an elbow-containing path through several housing elements. The stem 2 and the main body 3 substantially correspond to the embodiment mode of FIG. 1, though in this instance they are crossed by a functional duct proximally issuing into a stub 16. In this design, the image guide and the optic fiber run through the stem 2 and the main body 3, then through a tube 17 at a right angle to the main body 3, and next through an elbow 18 and a tube 19 into a terminal element 20 to which the imaging guide and the optic fiber are connected (not shown) by means of a camera. In the state of the art, the housing 15 of FIG. 4 is substantially composed of tube segments. In such a conventional design, it is an exceedingly cumbersome procedure to assemble the internal elements therein.

As regards the present invention, the housing 15 shown in FIG. 4 may be divided into two partial shells 21, 21′ at a cross-sectional surface subtending separation lines in the plane of the drawing, said partial shells being shown in FIG. 5. All internal components may be assembled in a first of the partial shells and then be sealed off by depositing the second partial shell on the first partial shell.

FIG. 6 shows the housing 15 of FIG. 4 being formed of four partial shells 22, 22′; 22″, 22′″, the four partial shells 22, 22′, 22″, 22′″ being divided along cross-sectional surfaces 23, 23′ and 23″ subtending separation lines, said surfaces being perpendicular to the plane of the drawing and also are shown in FIGS. 4 and 6.

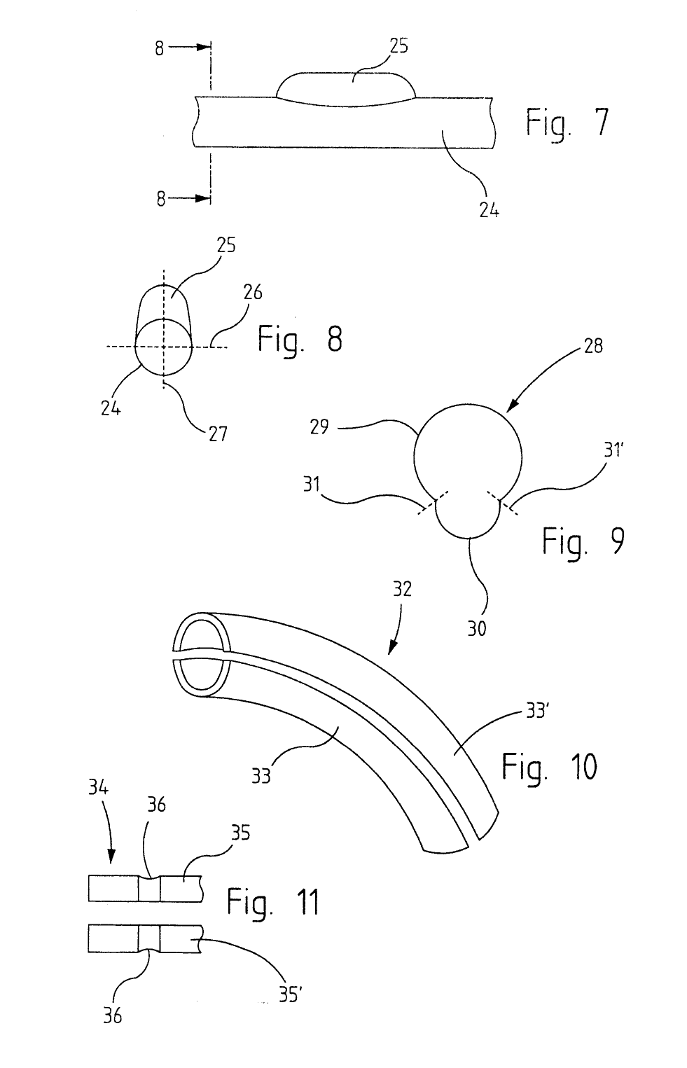

FIGS. 7 and 8 are a sideview and a cross-section of a tube segment 24 of a housing with a lateral (outward) bulge 25, respectively. Such a bulge could only be made with great difficulty in the state of the art by applying inside pressure to the straight tube segment 24. In the present invention, on the other hand, such a bulge may be made in a much easier manner by longitudinally dividing the tube segment along the separation lines at the cross-sectional surfaces 26 or 27 of FIG. 8. In both cases, partial shells are made very accurately and in a simple manner by pressure molding. The partial shells can then be joined to one another by welding.

FIG. 9 shows the cross-section of a housing tube 28 consisting of a peripheral position 29 of relatively large radius and of a peripheral portion 30 of relatively smaller radius. This cross-section is very difficult to make in the state of the art but it is easily made according to the invention in that the partial shells 29 and 30 are divided at cross-sectional surfaces 31 and 31′ where they can be joined to each other after the internal components have been inserted.

FIG. 10 shows a longitudinally divided tube 32 having a curving axis and having two partial shells 33 and 33′. These partial shells 33 and 33′ can be made in a compression mold, for instance, and thereby simplify and improve the manufacture as compared with bending a closed tube. Again this design simplifies emplacing the internal components considerably. It should be noted that the tube 32 of FIG. 10 may be a component of an outer endoscope housing and may contain an omitted tube which serves, for instance, as the system tube encompassing the image guide, such as a lens element(s) optics. This inner tube may also consist of partial shells to simplify manufacture and assembly of the particular lens element.

FIG. 11 shows the end portion of a tube 34 consisting of two partial shells 35, 35′. The tube 34 is fitted with a peripheral, impressed groove 36 which would be difficult to manufacture in a closed tube, but which is easily made by compression molding the partial shells 35, 35′. The impressed annular groove 36 may serve to hold an internal component such as the optics or the like. Illustratively, the light guide hookup stub 4 of the housing 1 of FIG. 1 in the partial shell division of FIG. 2 or 3 may be fitted with the annular groove 36 of FIG. 11 to grip the proximal end of the optic fiber.

In the above shown embodiment modes, the housings are shown separated at the separation lines situated on cross-sectional surfaces running parallel to the axis of the housing. As shown by these Figures, the separations are simple to implement. The separation lines, however, may run in a manner not explicitly shown above. For example, the separation lines can run obliquely or in a curving manner, so as to attain special effects. Illustratively, an arcuate separation line might allow a housing element that, per se, would rest on one partial shell instead of on the other partial shell.

1. A metal endoscope housing designed with longitudinal segments of different cross-sections and/or with a longitudinal axis which may follow right angles or be arcuate,
wherein the housing comprises separate semi-tubes which are separated at separation lines running substantially parallel to the longitudinal axis and which are joined in sealed manner to the separation lines.

2. A housing as claimed in claim 1, wherein the separation lines are connected by laser welding.

3. A housing as claimed in claim 1, wherein the semi-tubes are made by compression molding a sheetmetal.

4. A housing as claimed in claim 1, wherein the separation lines run parallel the housing's longitudinal axis.

5. A housing as claimed in claim 1, wherein the separation lines are configured to divide midway the housing cross-section.
